(19) United States
(12) Patent Application Publication
(10) Pub. No.: US 2006/0270898 A1
(43) Pub. Date: Nov. 30, 2006

(54) Penile girth enhancing condom

(76) Inventor: Charlotte Grace, Milwaukee, WI (US)

Correspondence Address: RYAN KROMHOLZ & MANION, S.C., POST OFFICE BOX 26618, MILWAUKEE, WI, 53226, US

(21) Appl. No.: 11/436,258

(22) Filed: May 18, 2006

Related U.S. Application Data
(60) Provisional application No. 60/682,303, filed on May 18, 2005.

(51) Int. Cl. A61F 5/00; A61F 6/02; A61F 6/04

(52) U.S. Cl. 600/38; 600/39; 128/842; 128/844

(57) ABSTRACT

A penile girth enhancing condom including at least one flexible rib member formed on the outer wall surface of the condom, the rib member extends along at least a majority of the length of the condom and provides added thickness to the condom wall. Alternatively, multiple flexible rib members are formed on the outer surface of the condom, which are circumferentially spaced.

### RELATED APPLICATION

This application claims the benefit of U.S. Provisional Application Ser. No. 60/682,303, filed May 18, 2005.

### BACKGROUND

The present invention relates generally to condoms. More specifically, the present invention relates to penile condoms used to prevent unwanted pregnancies and sexually transmitted diseases, and in addition, the condom may be used to add overall penile girth to thereby alter the overall sexual experience.

Condoms have been used for centuries. While early condom attempts were made with woven fabric, the first effective condoms were created from animal membranes. With the advent of vulcanization, rubber became the preferred condom material in the middle of the nineteenth century. In the 1930's, significant condom advancements were made in performance, comfort and affordability through the use of latex.

Currently, many different types of penile condoms are available. Some condoms may provide prosthetic assistance while others aim at preventing pregnancy and/or the transmission of sexually transmitted diseases. However, present condoms do not provide noticeable penile girth enhancement while at the same time permitting user sensitivity along its length. The present invention is directed toward a condom that seeks to enhance penile girth while at the same time functioning to prevent pregnancy and/or the transmission of sexually transmitted diseases.

### BRIEF SUMMARY

The present invention is directed to an improved male condom. The condom includes a flexible, sheath-like tubular body that may be manufactured of a material known in the art. The body is comprised of a wall having a closed end, an open end, an inner wall surface and an outer wall surface. The present invention provides penile girth enhancement in the form of at least one rib, or raised area, positioned on the outer wall surface. The rib preferably extends longitudinally of a central axis and along at least a substantial portion of a longitudinal length of the body. The rib is configured to provide an area of relatively increased thickness to the condom wall, thereby also effectively increasing the overall penile girth of the wearer. The rib, or raised area, is formed from a substantially homogenous, flexible material and may have a relatively smooth surface. As is known, a reservoir or tip for collected semen is positioned at the closed end.

In an alternative embodiment, the condom includes a plurality of ribs or raised areas of increased thickness. These ribs may be circumferentially spaced and radially extending from the condom body. The circumferential spacing provides alternating areas of greater and lesser thicknesses.

In another alternative embodiment, the ribs or raised areas of the present invention may have a varied texture. The variation may be in the form of variation in the width or texture of the ribs or raised areas, as will be discussed in greater detail.

### BRIEF DESCRIPTION OF THE DRAWINGS

FIG. 1 is a perspective view of an embodiment of an improved condom according to the present invention.

FIG. 2 is an end elevation view of the condom illustrated in FIG. 1 and showing four raised rib areas.

FIG. 3 is a cross sectional view of the condom illustrated in FIG. 1 and taken along lines 3-3 thereof.

FIG. 4 is an end elevation view of an embodiment of a condom according to the present invention, similar to that of FIG. 2, but showing five raised rib areas.

FIG. 5 is an end elevation view of an embodiment of a condom according to the present invention, similar to that of FIGS. 2 and 4, but showing six raised rib areas.

FIG. 6 is a side elevation view of an embodiment of a condom according to the present invention, and showing raised rib areas having a wavy profile.

FIG. 7 is a side elevation of an embodiment of a condom according to the present invention, and showing raised rib areas having an outer surface with upstanding protrusions.

### DESCRIPTION OF THE PREFERRED EMBODIMENT

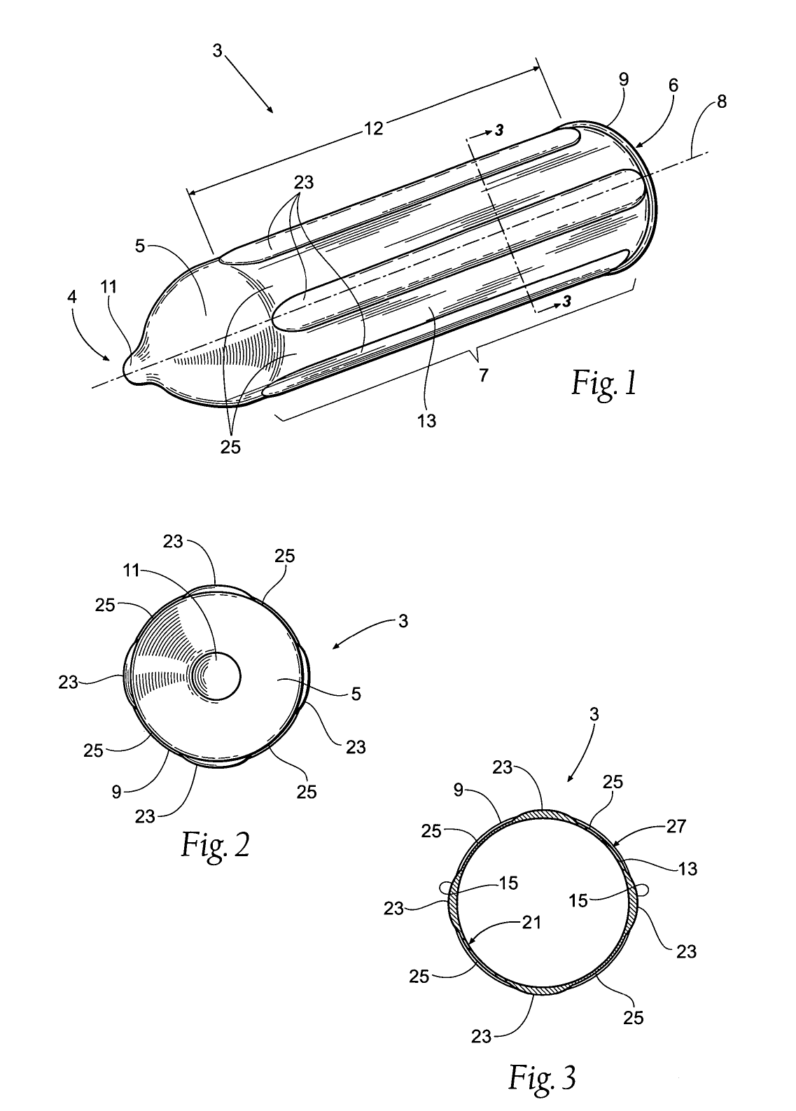

Referring now to FIGS. 1-3, an embodiment of the improved condom 3 may be seen. As illustrated, the condom 3 includes a flexible, sheath-like tubular body 7 that is configured to cover a penis (not shown). The tubular body 7 is comprised of a wall 13 having a closed distal end 4, which includes tip portion 5, and an open proximal end 6, which includes base ring portion 9. As seen particularly in FIG. 3, the wall 13 includes an inner surface 21 and an outer surface 27, wherein the outer surface 27 includes at least one rib, or raised area 23. The rib 23 extends longitudinally of a central axis 8 and along at least a substantial portion of a longitudinal length of the tubular body 7. The rib 23 is configured to provide an area of relatively increased thickness to the condom wall 13, thereby also effectively increasing the overall penile girth of the wearer. It is to be noted that the rib, or raised area 23, is preferably formed from a substantially homogenous, flexible material and having a relatively smooth surface 15.

In general, the improved condom 3 of the present invention is preferably formed from a prophylactically impermeable elastomeric material such as latex; however, other materials may be employed to the advantage of those users allergic to latex. Although described as different portions, the entire condom 3 is preferably formed contemporaneously through methods such as blow extrusion, latex resin dipping, polyurethane dipping, heated film and vacuum, heat sealing, or any other method known in the art or developed hereafter.

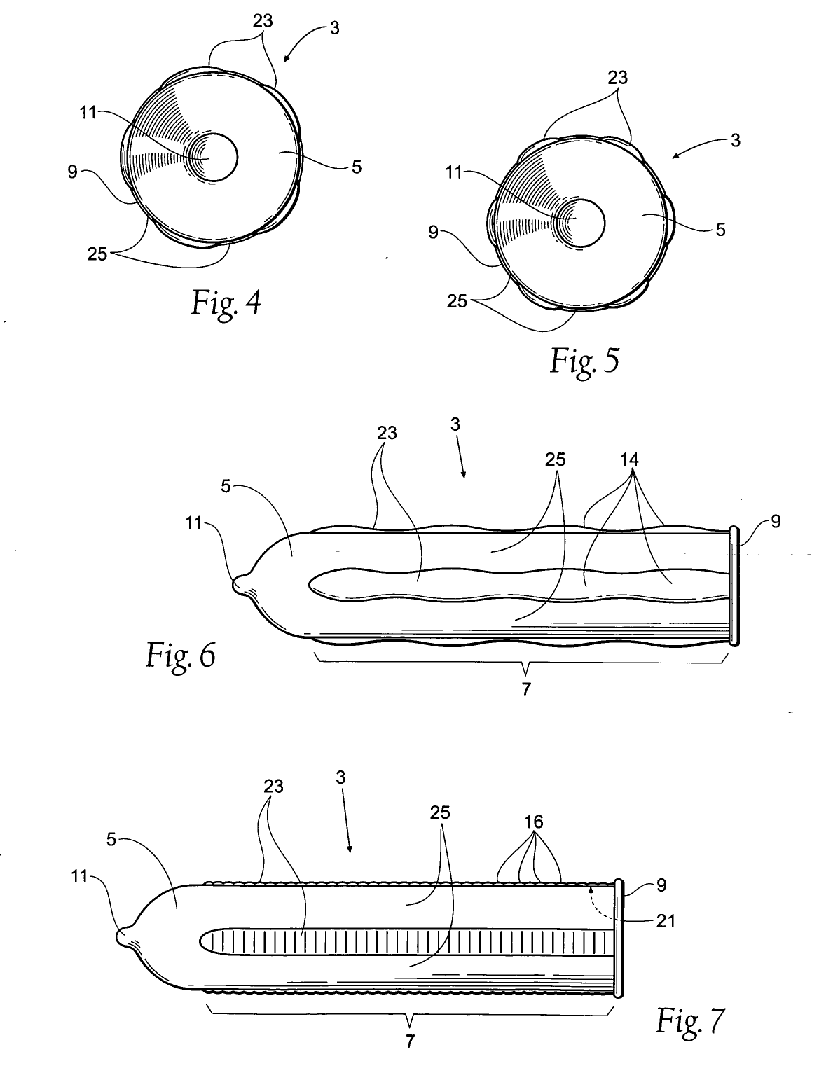

As may be further seen in FIGS. 1-7, the tip portion 5 is preferably of a generally domed shape, including a protruding section 11 that is commonly referred to as a reservoir tip. The reservoir tip 11 is well known in the art and serves to contain ejaculate. The tip portion 5 is preferably constructed of the same material as the body portion 7 of the condom 3, and the tip portion 5 preferably has a substantially uniform thickness approximating that of a standard condom (not shown).

As is illustrated particularly in the cross sectional view of FIG. 3, the sheath-like tubular body portion 7 includes a wall 13 having at least a first thickness 25 and a second thickness seen as a raised area rib 23. As seen, thickness varies between the inner wall surface 21 and the outer wall surface 27. Further, the body portion 7 is seen to have a longitudinal central axis 8 and a length 12. As seen in FIGS. 1 and 3, the tubular body portion 7 includes at least one rib, or raised area 23, on the outer surface 27. The rib 23 extends longitudinally of the central axis 8 and along at least a substantial portion of a longitudinal length 12 of the body portion 7. The rib 23 is configured to provide an area of relatively increased thickness to the condom wall 13, thereby also effectively increasing the overall penile girth of the wearer. The rib, or raised area of increased thickness 23, is preferably formed from a substantially homogenous, flexible material and having a relatively smooth surface 15. The rib, or raised area of increased thickness 23, serves to enhance the sexual experience by providing extra penile girth, thereby boosting the confidence of the user and enhancing the pleasure of the person with whom it is used. As is further seen, the wall 13 also includes areas of lesser thickness 25. It is preferred to have at least one area of lesser thickness 25 to thereby increase tactile sensation of the wearer.

Although at least one rib or raised area 23 is needed to practice the present invention, as shown in the Figures, a preferred embodiment includes a plurality of ribs or raised areas of increased thickness 23. As may be seen, the ribs 23 are preferably radially extending from the outer wall surface 27 and are circumferentially spaced around the sheath-like tubular body portion 7; however, spacing could be altered if desired. While any number of ribs 23 may be used up to and including the number necessary to encircle substantially the entire body portion 7, it is preferable allow for longitudinal, circumferentially located areas of lesser thickness 25 between the ribs of increased thickness 23. The areas of lesser thickness 25 provide a greater sensitivity to the user and his partner relative to the sensitivity provided by the ribs of increased thickness 23. It is contemplated that the thickness of the areas of lesser thickness 25 may approximate the thickness of a standard condom. The raised ribs 23 are preferably made of a sufficiently pliable, or flexible, material so as to allow ease in rolling the condom 3, as well as for user and partner comfort.

As may be seen in the Figures the condom 3 further preferably includes a base ring portion 9. The base ring portion 9 of the condom 3 has an inner diameter substantially equal to the inner diameter 10 of the body portion 7. The base ring technology is well known in the art. The ring 9 serves as a final support member that aids in retaining the condom shape.

Alternatively, and as seen particularly in FIGS. 5 and 6, the raised ribs 23 may include variation in outer surface texture and outline. For example, the ribs 23 may have various finishes, such the rolling bumps of varying width 14 illustrated in FIG. 5, or the upstanding protrusions 16, depicted in FIG. 6. As described in connection with the previous views, the ribs 23 shown in the views of FIGS. 5 and 6 are preferably radially extending from the outer wall surface 27 and are circumferentially spaced around the sheath-like tubular body portion 7. As discussed, spacing may be altered, if desired. While any number of ribs 23 may be used up to and including the number necessary to encircle substantially the entire body portion 7, it is preferable allow for longitudinal, circumferentially located areas of lesser thickness 25 between the ribs of increased thickness 23. The areas of lesser thickness 25 provide a greater sensitivity to the user and his partner relative to the sensitivity provided by the ribs of increased thickness 23.

The improved condom 3 is used in the manner typical to that of a standard rolled condom.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. An improved male condom comprising:
a flexible tubular body having a predetermined length and a longitudinal axis, the tubular body comprising a wall having a closed end, an open end, an inner wall surface, and an outer wall surface;
said wall having a first predetermined thickness and a second predetermined thickness, wherein said first predetermined thickness is relatively greater than said second thickness; and
said wall including at least one rib member radially extending from said outer wall surface, said rib extending longitudinally of and substantially parallel to said longitudinal axis and extending along at least a majority of a longitudinal portion of said tubular body, said rib member being configured to provide said first predetermined thickness to the wall of said condom.

2. The condom of claim 1 wherein said rib member is comprised of a substantially homogeneous, flexible material.

3. The condom of claim 1 wherein said rib member includes an outer surface.

4. The condom of claim 3 wherein said outer surface is substantially smooth.

5. The condom of claim 1 further including a plurality of rib members circumferentially spaced around said tubular body and radially extending from said outer wall surface.

6. A condom according to claim 3 wherein said outer surface of said rib member includes a plurality of upstanding protrusions.

7. A condom according to claim 1 wherein said rib member has a varying width.

8. A condom comprising:
a tubular body having a predetermined length surrounding a longitudinal axis, the tubular body having a closed end, an open end, and an outer surface;
a plurality of circumferentially spaced, longitudinally extending flexible rib members, said rib members radially extending from said outer surface of said tubular body; and
wherein said rib members are comprised of a substantially homogeneous material.

9. A condom according to claim 8 wherein said rib members extend substantially parallel to the longitudinal axis and along at least a majority of the length of the tubular body.

10. A condom according to claim 8 wherein said rib members are equally circumferentially spaced and including longitudinally extending areas of lesser thickness between said rib members.

11. A condom according to claim 8 wherein at least one rib member includes an outer surface having a plurality of upstanding protrusions.

12. A condom according to claim 8 wherein at least one rib member has a varying width.

13. A condom according to claim 8 wherein at least one rib member has a varying thickness.
